(19) United States
(12) Patent Application Publication
(10) Pub. No.: US 2014/0017129 A1
(43) Pub. Date: Jan. 16, 2014

(54) OXIDATION METHOD AND OXIDATION APPARATUS OF SULFUR COMPOUNDS IN SAMPLE GAS AND ANALYSIS APPARATUS FOR SULFUR COMPOUNDS

(71) Applicant: TAIYO NIPPON SANSO CORPORATION, Tokyo (JP)

(72) Inventors: Yusuke Miki, Tokyo (JP); Yasuo Hirose, Tokyo (JP)

(21) Appl. No.: 13/938,474

(22) Filed: Jul. 10, 2013

(30) Foreign Application Priority Data
Jul. 13, 2012 (JP) 2012-157160
Feb. 22, 2013 (JP) 2013-032769

(51) Int. Cl. G01N 33/00

(52) U.S. Cl. CPC G01N 33/0013
USPC 422/83; 423/242.1; 422/129

(57) ABSTRACT

Provided is an oxidation method and oxygen apparatus of sulfur compounds in a gas in which stable sulfur compounds such as carbonyl sulfide can be easily converted into sulfur oxide, and an analysis apparatus of sulfur compounds to which the oxidation method and the oxidation apparatus are applied. Sulfur compounds other than sulfur dioxide contained in a gas is subjected to a silent discharge treatment, whereby those sulfur compounds are oxidized and converted into sulfur dioxide. The analysis apparatus includes a silent discharge treatment unit in which a gas containing sulfur compounds is subjected to a silent discharge treatment to oxidize sulfur compounds other than sulfur dioxide to be converted into sulfur dioxide, and an analyzing unit in which the concentration of sulfur dioxide contained in the gas which has been subjected to silent discharge treatment in the silent discharge treatment unit is measured.

### FIELD

The present invention relates to an oxidation method and an oxidation apparatus of sulfur compounds in a gas and an analysis apparatus for sulfur compounds, and more specifically, relates to an oxidation method and an oxidation apparatus in which sulfur compounds contained in a variety of gases are oxidized into sulfur dioxide, and an analysis apparatus for sulfur compounds in a sample gas to which the oxidation method and the oxidation apparatus are applied.

### BACKGROUND

For the purpose of analyzing the concentration of a variety of sulfur compounds such as hydrogen sulfide or a sulfurous acid gas (sulfur dioxide) which are harmful components in the air existing as impurities in a variety of gases, a variety of analysis methods and analysis apparatuses have been conventionally proposed. For example, known is a method in which sulfur compounds such as hydrogen sulfide are allowed to react with ozone to measure the concentration (for example, see Patent Document 1), or a method in which a gas containing sulfur dioxide is irradiated with ultraviolet to selectively measure the concentration of sulfur dioxide (for example, see Patent Document 2).

### PRIOR ART DOCUMENTS

### Patent Documents

[Patent Document 1] Japanese Published Unexamined Patent Application No. 2005-3585

[Patent Document 2] Japanese Published Unexamined Patent Application No. 2004-138466

### SUMMARY

### Problem to be Solved by the Invention

By the way, in order to measure the concentration of sulfur contained in a variety of sulfur compounds other than hydrogen sulfide and sulfur dioxide, the sulfur compounds other than hydrogen sulfide and sulfur dioxide are needed to be converted into hydrogen sulfide or sulfur dioxide. However, it has been difficult to convert sulfur compounds having stable structures with multiple bonds such as carbonyl sulfide (COS) into sulfur dioxide with a conventional oxidation or reduction method such as an oxidation method using ozone.

Accordingly, the present invention is aimed at providing an oxidation method and oxidation apparatus of sulfur compounds in a gas in which stable sulfur compounds such as carbonyl sulfide can be easily converted into sulfur oxide, and an analysis apparatus of sulfur compounds to which the oxidation method and the oxidation apparatus are applied.

### Means for Solving the Problem

In order to attain the above-mentioned object, the oxidation method of sulfur compounds in a sample gas of the present invention is an oxidation method of sulfur compounds in which sulfur compounds other than sulfur dioxide contained in a sample gas are oxidized and converted into sulfur dioxide wherein the sample gas is subjected to a silent discharge treatment, whereby sulfur compounds other than the sulfur dioxide are oxidized and converted into sulfur dioxide. During the silent discharge treatment, oxygen and argon are preferably added as auxiliary gases to the sample gas. Alternatively, before the silent discharge treatment, the sample gas is preferably introduced into pipe formed by oxygen permeable materials.

The oxidation apparatus for sulfur compounds in a sample gas of the present invention is oxidation apparatus of sulfur compounds in which sulfur compounds other than sulfur dioxide contained in a sample gas are oxidized and converted into sulfur dioxide, comprising a pipe formed by oxygen permeable materials in which the sample gas is introduced and a silent discharge treatment unit in which a sample gas emitted from the pipe is subjected to a silent discharge treatment.

Further, the analysis apparatus of sulfur compounds of the present invention is an analysis apparatus for measuring the concentration of sulfur compounds contained in a gas, comprising a silent discharge treatment unit in which a gas containing sulfur compounds is subjected to a silent discharge treatment to oxidize sulfur compounds other than sulfur dioxide contained in the gas to be converted into sulfur dioxide, and an analyzing unit in which the concentration of sulfur dioxide contained in the gas which has been subjected to silent discharge treatment in the silent discharge treatment unit is analyzed. Further, the analysis apparatus of sulfur compounds of the present invention preferably comprises an auxiliary gas addition unit for adding oxygen and argon to the gas which is introduced to the silent discharge treatment unit as auxiliary gases. Alternatively, a pipe by which a sample gas is introduced into the silent discharge treatment unit is preferably formed by oxygen permeable materials.

### Effect of the Invention

By the present invention, sulfur compounds (excepting sulfur dioxide, those that follow are the same) can be oxidized at high efficiency and converted into sulfur dioxide by employing a simple device configuration, and an analysis of the total sulfur contained in a gas to be a sample can be easily and precisely performed by analyzing the concentration of total sulfur dioxide including the converted sulfur dioxide. In addition, by adding oxygen or argon as an auxiliary gas, silent discharge is likely to occur, and oxygen atoms which oxidize sulfur compounds can be sufficiently generated.

By using, as the oxygen source at the time of oxidizing sulfur compounds by silent discharge treatment, oxygen permeated from a pipe formed by oxygen permeable materials, oxidation can be performed without mixing a gas for oxygen addition at the time of oxidation treatment, and total sulfur in a sample gas can be easily measured. By appropriately setting the flow rate, or the pressure of a sample gas introducing to the pipe, the amount of oxygen which permeates the pipe can be adjusted in an optimal amount.

### BRIEF DESCRIPTION OF THE DRAWINGS

FIG. 1 is an explanatory drawing illustrating a first embodiment of a silent discharge treatment unit for carrying out an oxidation method of sulfur compounds in a gas of the present invention.

FIG. 2 is an explanatory drawing illustrating a first embodiment of an analysis apparatus of the present invention using an oxidation apparatus to which the oxidation method of sulfur compounds in a gas of the present invention is applied.

FIG. 3 is an explanatory drawing of an analysis apparatus representing one example of a device configuration when generating a calibration curve of sulfur compounds to be analyzed.

FIG. 4 is an explanatory drawing illustrating one example of conventional analysis apparatus using an oxidation apparatus in which oxidation of sulfur compounds excepting sulfur dioxide is performed by ozone.

FIG. 5 is a diagram illustrating a result of analysis of carbonyl sulfide.

FIG. 6 is a diagram illustrating a result of analysis of a variety of sulfur compounds.

FIG. 7 is a calibration curve illustrating the relationship between the carbonyl sulfide concentration and the sulfur dioxide concentration.

FIG. 8 is an explanatory drawing illustrating a second embodiment of an analysis apparatus of sulfur compounds in a sample gas to which the present invention is applied.

FIG. 9 is an explanatory drawing of an experimental apparatus in which oxygen permeability in Experimental Example 1 and Experimental Example 2 is confirmed.

FIG. 10 is a diagram illustrating change in oxygen concentration obtained in Experimental Example 1.

FIG. 11 is a diagram illustrating change in oxygen concentration obtained in Experimental Example 2.

FIG. 12 is an explanatory drawing of an experimental apparatus used in Experimental Example 3.

FIG. 13 is a diagram illustrating peaks obtained in Experimental Example 3.

FIG. 14 is a diagram illustrating the relationship between the concentration of carbonyl sulfide provided and the concentration of sulfur dioxide measured in Example 1.

### DESCRIPTION OF EMBODIMENTS

An oxidation method of sulfur compounds in a gas as illustrated in a first embodiment can be carried out by performing a silent discharge treatment using a hermitically sealed silica glass tube for silent discharge 11 as illustrated in FIG. 1. The silica glass tube for silent discharge 11 is provided with a gas inlet unit 12 and a gas outlet unit 13, a cylindrical electrode 14 provided on the wall of a silica glass tube, an internal electrode 15 arranged in the axis direction of the cylindrical electrode 14, and a power source unit 16 which applies a direct current high voltage between the cylindrical electrode 14 and the internal electrode 15.

By applying a high voltage between the cylindrical electrode 14 and the internal electrode 15 which are covered with an insulator (dielectric substance), for example, a high voltage of 7 kV or higher is applied between both the electrodes 14, 15 when the distance between both the electrodes 14, 15 is 3 mm while flowing a gas containing oxygen in the silica glass tube for silent discharge 11, silent discharge occurs between both electrodes, and oxygen flowing in the pipe serves as an oxygen atom source to oxidize a variety of components contained in a gas. Accordingly, when sulfur compounds are contained in a gas flowing in the pipe, sulfur compounds other than sulfur dioxide can be oxidized and converted into sulfur dioxide.

In order to surely oxidize sulfur compounds other than sulfur dioxide in a gas (sample gas) by using the thus formed silica glass tube for silent discharge 11, it is preferable that an auxiliary gas for silent discharge to facilitate the occurrence of silent discharge of argon, helium or the like in the silica glass tube for silent discharge 11, and an auxiliary gas for oxidation containing oxygen for the generation of oxygen atom be introduced together with the sample gas.

It is preferable that the auxiliary gas for silent discharge is introduced such that the percentage thereof be 50% (volume %, those that follow are the same) or higher in the silica glass tube for silent discharge 11. The gas to be used is preferably argon from the economic standpoint. For the auxiliary gas for oxidation, any gas may be used as long as the gas has an oxygen content by which needed oxygen atoms for oxidizing sulfur compounds other than sulfur dioxide contained in a sample gas can be provided, and normally, oxygen gas may be used.

Optimal amounts of introduction (flow rate in the pipe) of the auxiliary gas for silent discharge and the auxiliary gas for oxidation may be appropriately selected depending on a variety of conditions such as the properties of the sample gas, the concentration of sulfur compounds in the sample gas, the introduction amount of the sample gas, the structure of the silica glass tube for silent discharge 11 such as the gap between the electrodes, the length thereof and applied voltage. Depending on the conditions of silent discharge, not sulfur dioxide but sulfur monoxide or sulfur trioxide (sulfuric anhydride) may be generated. There is no problem with sulfur monoxide because sulfur monoxide is oxidized by an oxygen atom to become sulfur dioxide in a short time. Sulfur trioxide is very hardly to be generated compared with sulfur dioxide, which also does not affect the analysis.

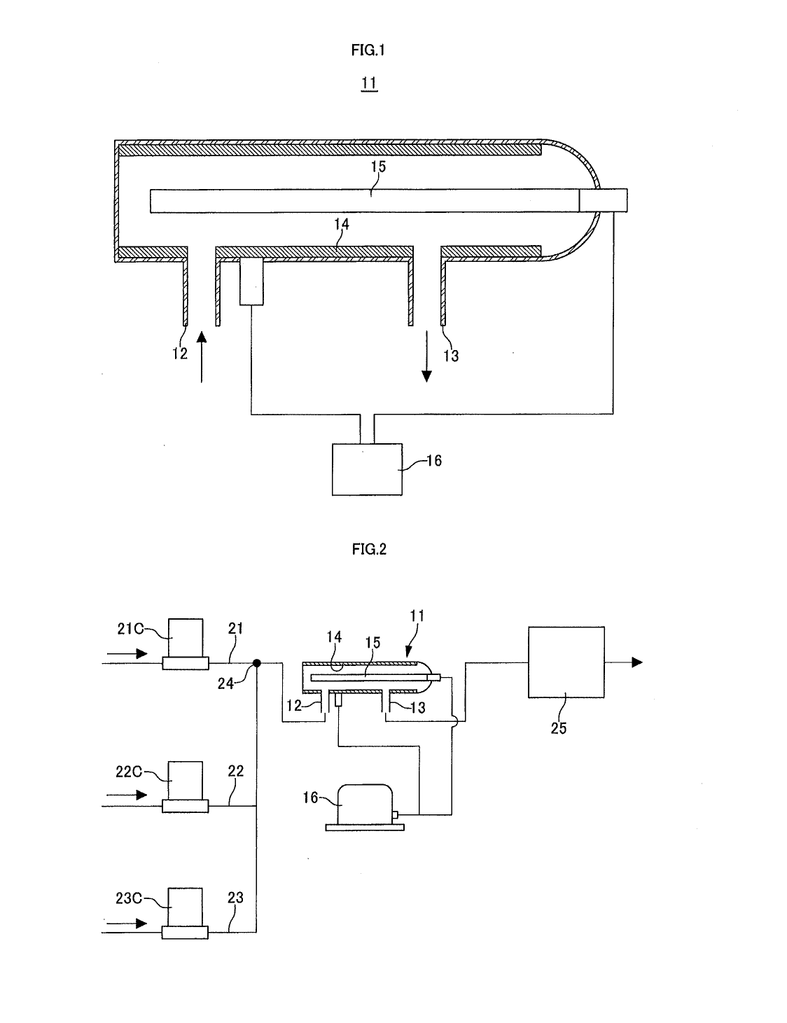

FIG. 2 illustrates a first embodiment of an analysis apparatus of the present invention for analyzing the concentration of sulfur compounds in a sample gas by using the silica glass tube for silent discharge 11. In the present embodiment, the silica glass tube for silent discharge 11 which is a silent discharge treatment unit is formed such that, to the gas inlet unit 12 of the silica glass tube for silent discharge 11, a sample gas introduction channel 21, an argon introduction channel 22 and an oxygen introduction channel 23 provided with a mass flow controller (MFC) 21C, 22C, 23C, respectively are provided, and such that a sample gas from the sample gas introduction channel 21, argon from the argon introduction channel 22 and oxygen from the oxygen introduction channel 23 merge at a merging unit 24 to be mixed and introduced into the silica glass tube for silent discharge 11. To a gas outlet unit 13, an analyzer 25 which can analyze sulfur dioxide in a gas is connected as an analyzing unit. When oxygen is sufficiently contained in a sample gas to be measured, introduction of oxygen can be omitted.

The oxidation method and analyzing method of sulfur compounds using the thus formed analysis apparatus is as mentioned below. After mixing a sample gas containing sulfur compounds to be analyzed with a predetermined percentage of argon and oxygen as auxiliary gases at the merging unit 24, the mixture is introduced into the silica glass tube for silent discharge 11, and at the same time, a direct current high voltage is applied between the cylindrical electrode 14 and the internal electrode 15 from the power source unit 16 to generate silent discharge between the electrodes 14, 15. By this, oxygen in the gas flowing in the pipe generates an oxygen atom, and a variety of sulfur compounds excepting sulfur dioxide contained in the sample gas are oxidized by the oxygen atom to be converted into sulfur dioxide. All sulfur dioxide including sulfur dioxide converted in the silica glass tube for silent discharge 11 is introduced into an analyzer 25 to be analyzed. By performing a predetermined processing, the total sulfur concentration obtained by summing sulfur components of a variety of sulfur compounds contained in the sample gas can be calculated.

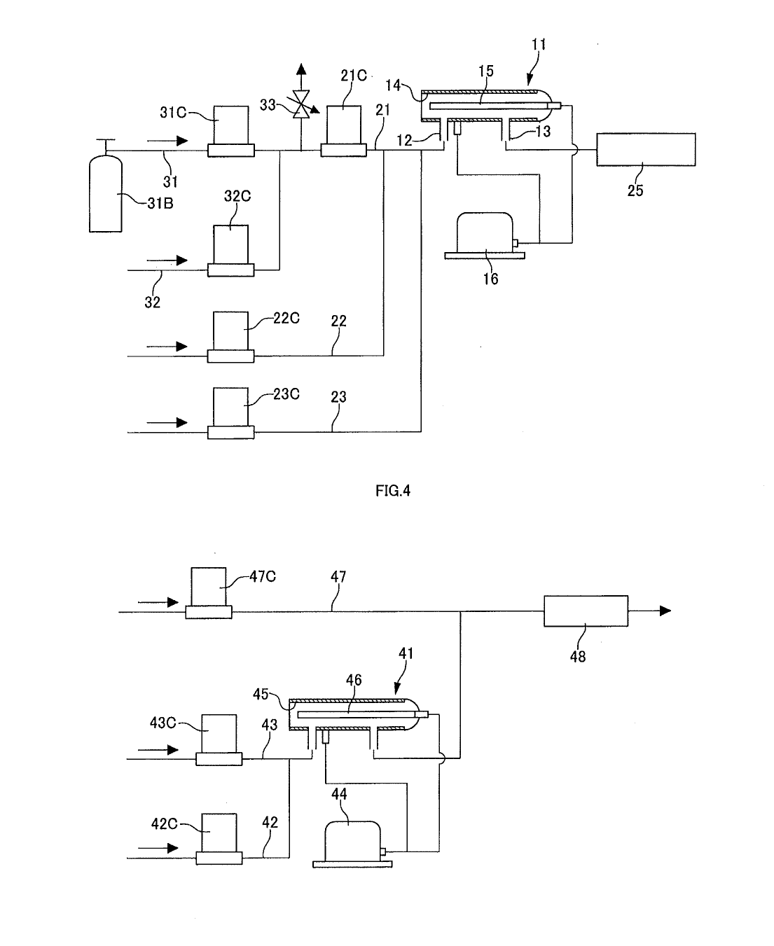

FIG. 3 illustrates a device configuration when a calibration curve of sulfur compounds to be analyzed is generated. In the following description, to the same component as that of the analysis apparatus as illustrated in the embodiment, the same reference numeral is provided and a detailed explanation thereof will be omitted. An apparatus for generating a calibration curve is formed such that, on the upstream of the sample gas introduction channel 21, a standard gas introduction channel 31 in which a standard gas is introduced from a standard gas container 31B filled with a standard gas containing sulfur compounds to be analyzed at a known concentration via a mass flow controller 31C, and a zero gas introduction channel 32 by which a zero gas which does not contain sulfur compounds to be analyzed and does not affect the analysis is introduced via a mass flow controller 32C are provided, and by controlling the mass flow controllers 31C, 32C, a mixed gas which is adjusted at a known sulfur compound concentration by mixing a standard gas and a zero gas can be introduced into the sample gas introduction channel 21. On the upstream of the mass flow controller 21C, a pressure regulator 33 for exhausting an excess gas and adjusting the pressure is provided.

For the analyzer 25, a variety of analyzers by which the concentration of sulfur dioxide can be measured can be used. For example, a commercially available ultraviolet fluorescence type sulfur dioxide analysis apparatus, mass spectrometer or flame photometric detector can be used. By arranging a separation column for gas chromatograph for component separation before a separation analyzer 25, qualitative analysis of sulfur compounds can also be performed. When separation is performed, a pretreatment such as precut or concentration can be combined. Further, when sulfur compounds are oxidized by silent discharge, the type and the structure of apparatus for performing a silent discharge treatment can be arbitrarily selected.

### EXAMPLE 1

By using an analysis apparatus having a configuration as illustrated in FIG. 2, carbonyl sulfide was analyzed. For reference, by using an analysis apparatus intended for a conventional contact oxidation method having a configuration as illustrated in FIG. 4, carbonyl sulfide was analyzed similarly.

A conventional analysis apparatus as illustrated in FIG. 4 has a configuration in which, to the silica glass tube for silent discharge 41 having the same configuration as that of silica glass tube for silent discharge 11 as illustrated in FIG. 1, oxygen which is an ozone source from the oxygen introduction channel 42 and argon which is a gas for discharging from an argon introduction channel 43 are introduced at a predetermined rate via the mass flow controllers 42C, 43C, respectively, and by applying a high voltage between the cylindrical electrode 45 and the internal electrode 46 from the power source unit 44, ozone is generated, and then a gas containing the generated ozone gas and a sample gas introduced form a sample gas introduction channel 47 via a mass flow controller 47C are mixed to be introduced to an analyzer 48.

For analyzers 25, 48 of both the analysis apparatuses, a gas chromatograph/flame photometric detector in which each sulfur compound is separated and a qualitative analysis is possible was used individually. For the sample gas, a gas whose carbonyl sulfide concentration was adjusted to 1 ppm was used individually. The same silica glass tube for silent discharge was used, and the applied voltage was the same. Further, the amount of gas introduced was the same.

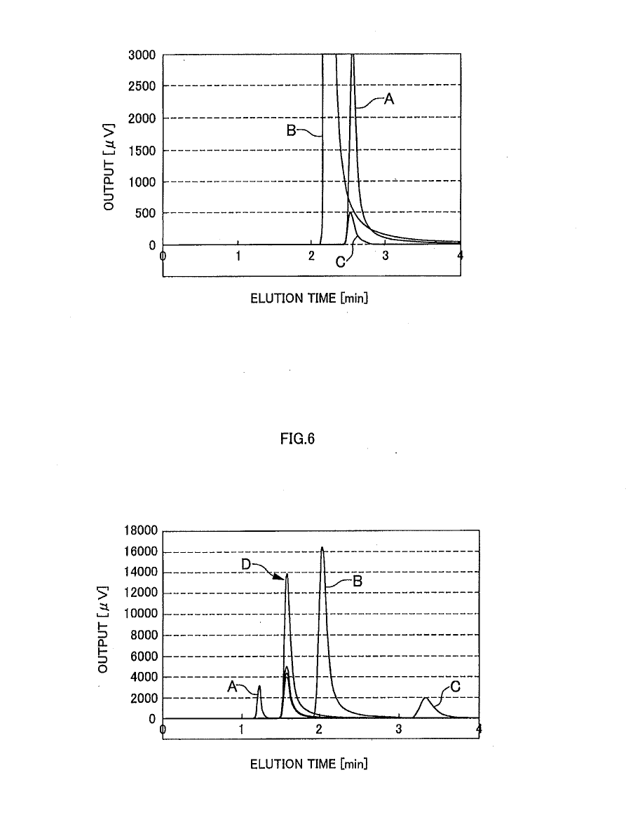

FIG. 5 shows the result of analysis of carbonyl sulfide. Peak A represents a peak detected when an oxidation method of sulfur compounds of the present invention was performed; peak B represents a peak detected when a conventional contact oxidation method was performed; and peak C represents a peak of a standard gas whose sulfur dioxide concentration was adjusted to 0.1 ppm. As illustrated in FIG. 5, by performing the oxidation method of sulfur compounds of the present invention, the peak of carbonyl sulfide disappears and a peak of sulfur dioxide appears, which shows that most of carbonyl sulfide is oxidized by a silent discharge treatment to be converted into sulfur dioxide. On the other hand, since, in the case of the contact oxidation method, a peak of sulfur dioxide does not appear at all, it is found that carbonyl sulfide is not oxidized by contact with ozone.

Further, by using both analysis apparatuses, the analyses of hydrogen sulfide, methyl mercaptan, dimethyl sulfide and sulfur dioxide were performed. As the result, while, as illustrated in FIG. 6, in the case of conventional contact oxidation method, peak A of hydrogen sulfide, peak B of methyl mercaptan and peak C of dimethyl sulfide appeared, in the case in which the oxidation method of sulfur compounds of the present invention is applied, it is found that all peaks overlap at peak portion D of sulfur dioxide.

From these results, by the oxidation method of the sulfur compound of the present invention, it is found that a variety of sulfur compounds can be oxidized and converted into sulfur dioxide. By this, a variety of sulfur compounds contained in the sample gas can be made into sulfur dioxide, and therefore, by combining an analyzer for analyzing a commercially available sulfur dioxide, the total sulfur concentration in a variety of gases can be easily and precisely analyzed.

### EXAMPLE 2

An analysis of carbonyl sulfide was performed in a similar manner as in Example 1 except that the analyzer in Example 1 was replaced with a gas chromatograph mass spectrometer. In the mass spectrometry, for sulfur dioxide, peaks appeared at mass numbers of 48 and 64; and for carbonyl sulfide, a peak appeared at a mass number of 60. As listed on Table 1, for a standard gas whose sulfur dioxide concentration was 5 ppm, distinctive peaks appeared at mass numbers of 48 and 64 individually. In a conventional contact oxidation method, a peak of carbonyl sulfide appeared at a mass number of 60, and with respect to the peak area of carbonyl sulfide, the peak areas of sulfur dioxide of mass numbers 48 and 64 were very small. On the other hand, when an oxidation method of sulfur compounds of the present invention is applied, the peak area of sulfur dioxide is increased and the peak area of the carbonyl sulfide becomes small. Accordingly, by applying an oxidation method of sulfur compounds of the present invention, it is found that carbonyl sulfide is converted into sulfur dioxide.

TABLE 1

| | M/Z | | | | | |
|---|---|---|---|---|---|---|
| | 48 | | 60 | | 64 | |
| Item | Area | Height | Area | Height | Area | Height |
| Standard gas | 627 | 48 | No peak | | 940 | 69 |
| Sulfur dioxide: 5 ppm | | | | | | |
| Carbonyl sulfide | 2 | 0.2 | 877 | 80 | 2 | 0.2 |
| (Conventional | | | | | | |
| apparatus) | | | | | | |
| Carbonyl sulfide | 45 | 3 | 6 | 0.7 | 73 | 5 |
| (present invention) | | | | | | |

### EXAMPLE 3

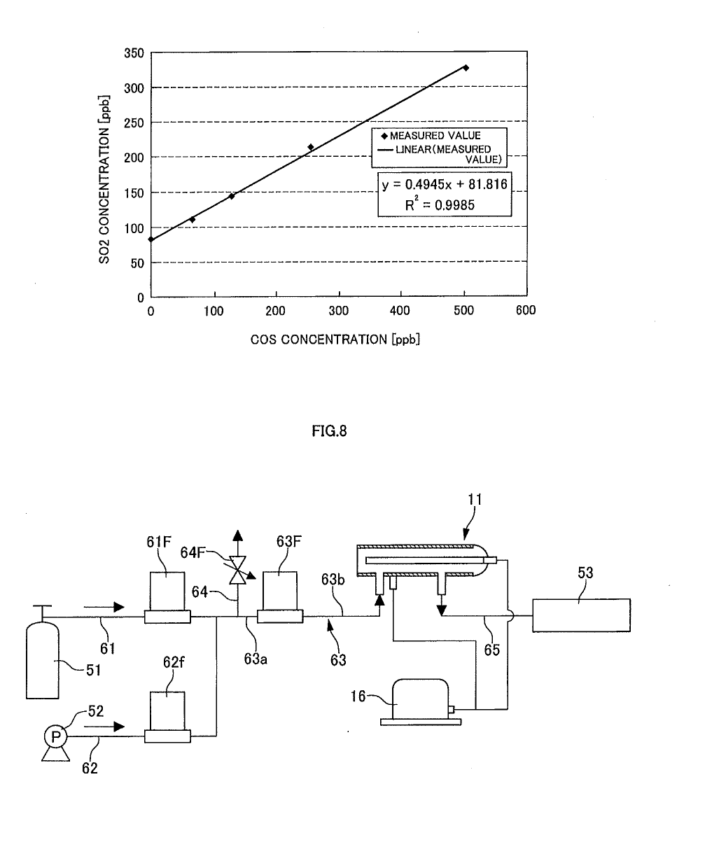

By using an analysis apparatus having a configuration as illustrated in FIG. 3, calibration curves for a variety of sulfur compounds were generated. For the analyzer 25, an ultraviolet fluorescence type spectrometer was used, and calibration curves of sulfur dioxide, carbonyl sulfide, hydrogen sulfide, methyl mercaptan, dimethyl sulfide and dimethyl disulfide after performing an oxidation treatment by silent discharge of the present invention were generated. Letting the measurement values for zero gas which does not contain each sulfur compound be zero points, the inclination, the zero point, the correlation function of each calibration curve are listed on Table 2. The calibration curve of carbonyl sulfide is illustrated in FIG. 7.

TABLE 2

| | | | Correlation |
|---|---|---|---|
| Component | Inclination | Zero point (ppb) | coefficient (R2) |
| Sulfur dioxide | 1.48 | 128 | 0.995 |
| Carbonyl sulfide | 0.49 | 82 | 0.998 |
| Hydrogen sulfide | 1.51 | 98 | 0.996 |
| Methyl mercaptan | 1.58 | 117 | 0.995 |
| Dimethyl sulfide | 1.34 | 89 | 0.991 |
| Dimethyl disulfide | 2.68 | 112 | 0.999 |

FIG. 8 illustrates a second embodiment of an analysis apparatus of sulfur compounds in a sample gas of the present invention to which an oxidation method of sulfur compounds in the sample gas and the oxidation apparatus of the sulfur compound in the sample gas in the present invention are applied. In the following description, to the same component as that of the analysis apparatus as illustrated in the embodiment, the same reference numeral is provided and a detailed explanation thereof will be omitted.

As illustrated in FIG. 8, the analysis apparatus illustrated in the present embodiment comprises as main devices a sample gas container 51 which is a sample gas source, an auxiliary gas source 52 for providing a purge gas or diluting gas, a silica glass tube for silent discharge (silent discharge treatment unit) 11 composed of a cylinder container made of silica glass, a power source unit 16 for providing power for discharging to the silica glass tube for silent discharge 11, and an analyzer 53 which is an analyzing unit for analyzing sulfur dioxide.

On a sample gas, pipe 61 in which a sample gas from the sample gas container 51 flows and an auxiliary gas pipe 62 in which an auxiliary gas from the auxiliary gas source 52 flows, flow control devices (mass flow controller (MFC)) 61F, 62F for accurately adjusting the flow rate of each gas are provided, and on a discharge treatment inlet side pipe 63 to which the sample gas pipe 61 and the auxiliary gas pipe 62 merge and which leads to the silica glass tube for silent discharge 11, a flow control device (mass flow controller (MFC)) 63F for accurately adjusting the flow rate of sample gas is provided. Further, on a gas merging pipe 63a positioned on the upstream of the flow control device 63F on the discharge treatment inlet side pipe 63, a gas emission pipe 64 having a flow control valve 64F is provided in order to exhaust a surplus gas from the gas merging pipe 63a.

In the case of the present embodiment, by bringing the cylindrical electrode 14 and the internal electrode 15 in FIG. 1 close to each other as much as possible, and by making the voltage applied between the electrodes 14, 15 high, it becomes possible to efficiently perform oxidation of sulfur compounds without demanding a gas such as argon or helium for facilitating the occurrence of silent discharge. For example, when the gap between the electrodes 14, 15 is about 1.5 mm, the voltage applied between electrodes 14, 15 is preferably 10 kV or higher.

For the analyzer 53, any analyzer can be used as long as it can measure the concentration of sulfur dioxide in a gas introduced into the analyzer 53, and usually, a commercially available trace sulfur dioxide analyzer or a detector may be used. When a plurality of sulfur compounds are contained in the sample gas, the total sulfur concentration is to be measured. The pressure may be set arbitrarily depending on the state of the sample gas or the specifications of the trace sulfur dioxide analyzer. When the pressure is adjusted, a pressure regulator may be arranged at an appropriate position on each pipe.

In an analysis apparatus having such a configuration, a pipe formed of oxygen permeable materials is used for the discharge treatment unit introduction pipe 63b in order to mix oxygen to be excited in the silica glass tube for silent discharge 11 into the sample gas. For the oxygen permeable materials, an appropriate synthetic resin material having gas permeability can be used, and those which have little reactivity with sulfur compounds containing sulfur dioxide, which does not affect the analysis of the sulfur compounds, and which has an excellent durability can be selected and used. For example, polytetrafluoroethylene (PTFE), polyvinyl chloride or the like can be used. In particular, since polytetrafluoroethylene has sufficient permeability of oxygen and also has excellent resistance to chemicals, and also has an advantage that sulfur compounds containing sulfur dioxide is hardly adsorbed, polytetrafluoroethylene is optimum for the material for pipe used in the present invention. Although those which permeate a gas other than oxygen can also be used, for example, those which permeate a large amount of water should not be used if possible since sulfur dioxide and water may react.

Although the suitable diameter, thickness, length of the discharge treatment unit introduction pipe 63b composed of oxygen permeable materials may vary depending on the flow rate or pressure of the sample gas flowing in the pipe, for example, when a tube made of polytetrafluoroethylene is used, the inner diameter thereof may be about 1 to 1.5 mm, and the thickness thereof may be about 0.5 to 1 mm; and the pressure of the sample gas flowing in the pipe may be set to about 50 to 100 kPa and the flow rate may be set to about 100 to 200 cc per minute. Although the length of discharge treatment unit introduction pipe 63b may be arbitrary, when these conditions are satisfied, the length of about 1 m is sufficient, and all of the discharge treatment unit introduction pipe 63b may be used and a part thereof may be used. All or a part of other pipe may be formed of similar materials.

Further, the above-mentioned advantage that sulfur compounds containing sulfur dioxide is hardly to be adsorbed in the polytetrafluoroethylene is effective also for all the pipe in the analysis apparatus of sulfur compounds in a sample gas of the present invention, and all the pipe may be made of polytetrafluoroethylene. In this case, at the pipe on the upstream of the gas inlet unit 12 of the silica glass tube for silent discharge 11, prevention of adsorption of sulfur compounds on the inner surface of the pipe and taking-in of oxygen in the pipe are expected. At the pipe on the downstream side of the gas outlet unit 13, prevention of adsorption of sulfur compounds on the inner surface of the pipe is expected. By this, addition of oxygen into the sample gas can be more efficiently performed, and at the same time, analysis precision can be improved.

### EXAMPLE 4

### Experimental Example 1

An experimental apparatus as illustrated in FIG. 9 was used. The experimental apparatus is formed such that, on both ends of a commercially available polytetrafluoroethylene tube (inner diameter 1.5 mm, thickness 1 mm, length 1 m) 71, shutoff valves 72, 73 are provided, and at the same time, a metal bypass tube 75 on which a bypass valve 74 is provided is connected to bypass the polytetrafluoroethylene tube 71, and by opening and closing the shutoff valves 72, 73 and bypass valve 74, either the polytetrafluoroethylene tube 71 or the metal bypass tube 75 is selected whereby a gas can flow therein. For the supply gas source 76, a purified nitrogen gas was used, and the flow rate of the nitrogen gas was adjusted to 1000 cc per minute by mass flow controller 77. For the analyzer 78, a trace oxygen analyzer was used, and each of the oxygen concentrations in nitrogen gas which has passed through the polytetrafluoroethylene tube 71 and the metal bypass tube 75 was measured.

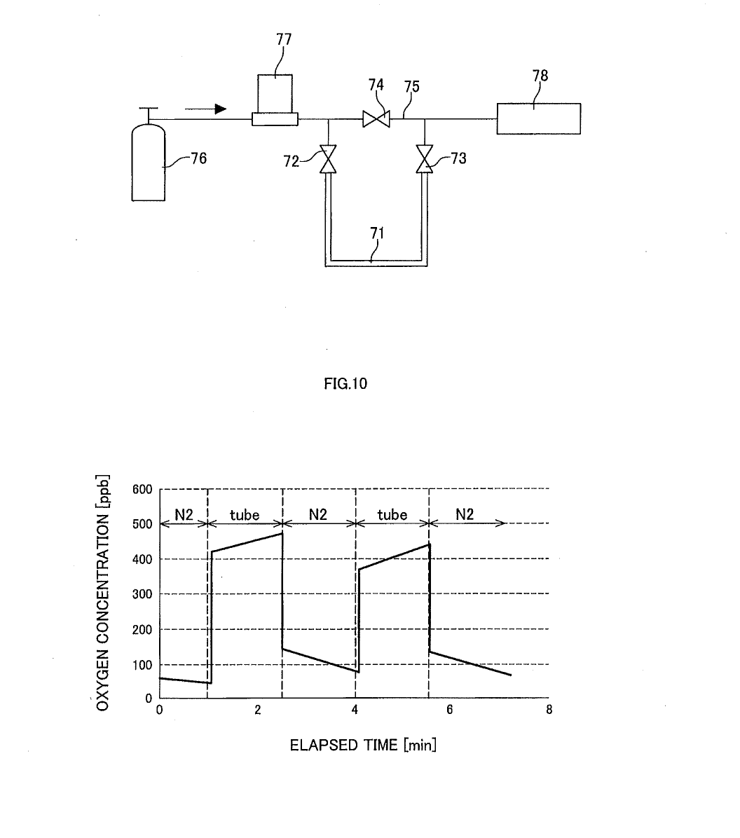

Change in the oxygen concentration at the time of closing shutoff valves 72, 73 and opening the bypass valve 74 (N2) and change in the oxygen concentration at the time of opening shutoff valves 72, 73 and closing the bypass valve 74 (Tube) are illustrated in FIG. 10. As the result, by flowing nitrogen gas into the polytetrafluoroethylene tube 71, it is found that oxygen in the air which permeated the polytetrafluoroethylene was mixed in nitrogen gas at about 400 to 500 ppb.

### Experimental Example 2

By using the same experimental apparatus as that in Experimental Example 1 as illustrated in FIG. 9, nitrogen gas was flowed in the polytetrafluoroethylene tube 71 while changing the flow rate at 0, 1000, 500, 250 cc per minute, and the oxygen concentration in nitrogen gas which passed through the polytetrafluoroethylene tube 71 was individually measured. The results are listed on FIG. 11. By these results, it was found that the flow rate of nitrogen gas flowing in the polytetrafluoroethylene tube 71 was in inverse proportion to the amount of oxygen mixed in the nitrogen gas, and since when the flow rate of the nitrogen gas is halved, the oxygen concentration doubles, the amount of oxygen in the air permeated the polytetrafluoroethylene per unit time is a constant rate.

### Experimental Example 3

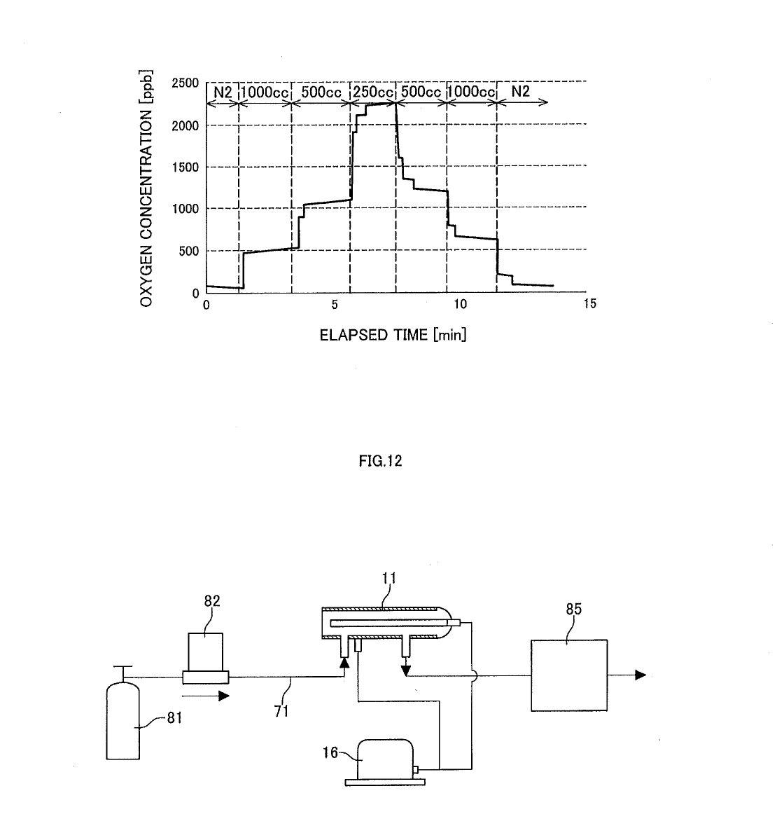

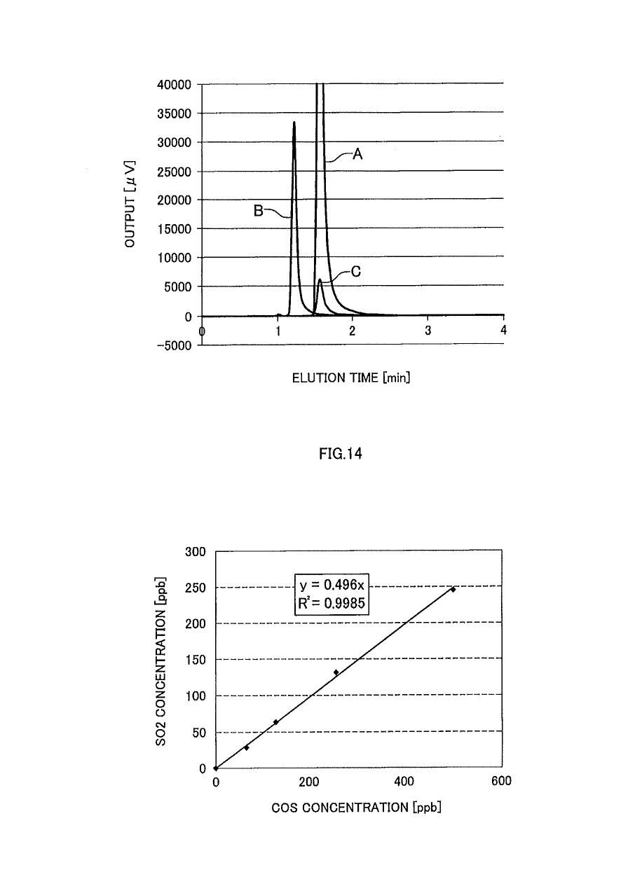

As illustrated in FIG. 12, a gas from a sample gas source 81 is controlled by a flow rate at a mass flow controller 82 to be introduced into the silica glass tube for silent discharge 11 to which 10 kV can be applied from the power source unit 16 via the same polytetrafluoroethylene tube 71 as that in Experimental Example 1 and the component of the gas emitted from the silica glass tube for silent discharge 11 was analyzed at GC-FPD (gas chromatograph with flame photometric detector) 85. For the sample gas source 81, sulfur dioxide, carbonyl sulfide are used; in cases where carbonyl sulfide was used, the case when the power source unit 16 was stopped and the case when power source unit 16 was operated were compared with each other. Peak (in the present example, peak at a mass number of 64) A of sulfur dioxide, peak (peak at a mass number of 60) B of carbonyl sulfide when the power source unit 16 was stopped and peak C when the power source unit 16 was operated and carbonyl sulfide was introduced are illustrated in FIG. 13.

From these results, it was found that, in cases where the power source unit 16 was operated and carbonyl sulfide was flowed, when carbonyl sulfide flowed in the polytetrafluoroethylene tube 71, oxygen in the air which permeated the polytetrafluoroethylene was excited by silent discharge at the silica glass tube for silent discharge 11, and carbonyl sulfide was converted into sulfur dioxide by the excited oxygen. Since when the power source unit 16 was operated, the peak of carbonyl sulfide does not appear, it was found that the total amount of introduced carbonyl sulfide was converted into other compounds such as sulfur dioxide, that there was a sufficient amount of oxygen for oxidizing carbonyl sulfide permeated into the polytetrafluoroethylene tube 71.

### Experimental Example 4

An analysis apparatus as illustrated in FIG. 8 was used, and for the sample gas container 51, a standard gas such as sulfur dioxide, carbonyl sulfide, hydrogen sulfide, methyl mercaptan, dimethyl sulfide and dimethyl disulfide was used; and for the auxiliary gas source 52, a purified nitrogen gas was used. By adjusting the flow rate of each gas at flow control devices 61F, 62F, the concentration of respective sample gases in nitrogen gas were adjusted to a plurality of known concentrations, and at the same time, the flow rate of the gas after adjusting the concentration at a flow control device 63F was adjusted. For the analyzer 53, a commercially available trace sulfur dioxide analyzer (manufactured by HORIBA, Ltd.) was used. For the discharge treatment unit introduction pipe 63, the same polytetrafluoroethylene tube 71 as that in Experimental Example 1 was used. In the silica glass tube for silent discharge 11, the gap between both ends of the electrodes 14, 15 was set to 1.5 mm; the applied voltage was set to 10 kV.

Each sample gas was diluted at a predetermined concentration by nitrogen gas to be provided, and the concentration of sulfur dioxide was measured at the analyzer 53. The relationship between the concentration of the provided carbonyl sulfide and the concentration of the measured sulfur dioxide is illustrated in FIG. 14. The inclination, correlation coefficient of the calibration curve of each of sample gases of sulfur dioxide, carbonyl sulfide, hydrogen sulfide, methyl mercaptan, dimethyl sulfide and dimethyl disulfide are listed on Table 3.

TABLE 3

| Component | Inclination | Correlation coefficient (R2) |
|---|---|---|
| SO₂ | 1.27 | 0.992 |
| COS | 0.59 | 0.995 |
| H₂S | 1.3 | 0.993 |
| CH₃SH | 1.27 | 0.992 |
| DMS | 1.13 | 0.988 |
| DMDS | 2.38 | 0.996 |

### DESCRIPTION OF THE REFERENCE NUMERALS

11 . . . Silica glass tube for silent discharge, 12 . . . Gas inlet unit, 13 . . . Gas outlet unit, 14 . . . Cylindrical electrode, 15 . . . Internal electrode, 16 . . . Power source unit, 21 . . . Sample gas introduction channel, 22 . . . Argon introduction channel, 23 . . . Oxygen introduction channel, 24 . . . Merging unit, 25 . . . Analyzer, 31 . . . Standard gas introduction channel, 31B . . . Standard gas container, 32 . . . Zero gas introduction channel, 33 . . . Control valve, 41 . . . Silica glass tube for silent discharge, 42 . . . Oxygen introduction channel, 43 ... Argon introduction channel, 44 . . . Power source unit, 45 . . . Cylindrical electrode, 46 . . . Internal electrode, 47 . . . Sample gas introduction channel, 48 . . . Analyzer, 21C, 22C, 23C, 31C, 32C, 42C, 43C, 47C . . . Mass flow controller, 51 . . . Sample gas container, 52 . . . Auxiliary gas source, 53 . . . Analyzer, 61 . . . Sample gas pipe, 62 . . . Auxiliary gas pipe, 63 . . . Discharge treatment inlet side pipe, 63a . . . Gas merging pipe, 63b . . . Discharge treatment unit introduction pipe, 61F, 62F, 63F . . . Flow control device (mass flow controller), 64 . . . Gas emission pipe, 64F . . . Flow control valve, 65 . . . Analyzer introduction pipe, 71 . . . Polytetrafluoroethylene tube, 72, 73 . . . Shutoff valve, 74 . . . Bypass valve, 75 . . . Metal bypass tube, 76 . . . Supply gas source, 77 . . . Mass flow controller, 78 . . . Analyzer, 81 . . . Sample gas source, 82 . . . Mass flow controller, 85 . . . GC-FPD

1. An oxidation method of sulfur compounds in which sulfur compounds other than sulfur dioxide contained in a sample gas is oxidized and converted into sulfur dioxide wherein the sample gas is subjected to a silent discharge treatment, whereby those sulfur compounds other than the sulfur dioxide are oxidized and converted into sulfur dioxide.

2. The oxidation method of sulfur compounds in a sample gas according to claim 1, wherein, when the silent discharge treatment is performed, oxygen and argon are added to the sample gas as auxiliary gases.

3. The oxidation method of sulfur compounds in a sample gas according to claim 1, wherein, before the silent discharge treatment, the sample gas is introduced in a pipe formed of oxygen permeable materials.

4. An oxidation apparatus of sulfur compounds in which sulfur compounds other than sulfur dioxide contained in a sample gas is oxidized and converted into sulfur dioxide, comprising a pipe formed by oxygen permeable materials in which the sample gas is introduced and a silent discharge treatment unit in which a sample gas emitted from the pipe is subjected to a silent discharge treatment.

5. An analysis apparatus for measuring the concentration of sulfur compounds contained in a sample gas, comprising a silent discharge treatment unit in which a gas containing sulfur compounds is subjected to a silent discharge treatment to oxidize sulfur compounds other than sulfur dioxide contained in the gas to be converted into sulfur dioxide, and an analyzing unit in which the concentration of sulfur dioxide contained in the gas which has been subjected to silent discharge treatment in the silent discharge treatment unit is analyzed.

6. The analysis apparatus of sulfur compounds according to claim 5, wherein the analysis apparatus of sulfur compounds comprises an auxiliary gas addition unit for adding oxygen and argon to the gas which is introduced to the silent discharge treatment unit as auxiliary gases.

7. The analysis apparatus of sulfur compounds according to claim 5, wherein a pipe by which a sample gas is introduced into the silent discharge treatment unit is formed by oxygen permeable materials.
